(19) United States
(12) Patent Application Publication
(10) Pub. No.: US 2017/0066801 A1
(43) Pub. Date: Mar. 9, 2017

(54) ALPHA3BETA HYDROGEN BOND SURROGATE MACROCYCLES AS MODULATORS OF RAS

(71) Applicants: Paramjit S. ARORA, Cold Spring Harbor, NY (US); Dafna BAR-SAGI, New York, NY (US); Stephen Taro JOY, Woodside, NY (US); Seth NICKERSON, Brooklyn, NY (US)

(72) Inventors: Paramjit S. ARORA, Cold Spring Harbor, NY (US); Dafna BAR-SAGI, New York, NY (US); Stephen Taro JOY, Woodside, NY (US); Seth NICKERSON, Brooklyn, NY (US)

(73) Assignee: NEW YORK UNIVERSITY, New York, NY (US)

(21) Appl. No.: 15/119,810

(22) PCT Filed: Feb. 23, 2015
(86) PCT No.: PCT/US15/17041

§ 371 Date: Aug. 18, 2016

Related U.S. Application Data
(60) Provisional application No. 61/943,363, filed on Feb. 22, 2014.
(60) Provisional application No. 61/943,339, filed on Feb. 22, 2014.

(51) Int. Cl. C07K 7/08

(52) U.S. Cl. CPC C07K 7/08; A61K 38/00

(57) ABSTRACT

The present invention relates to peptides having a stable, internally-constrained HBS α-helix, where the peptide mimics at least a portion of the α-H helix of the Sos protein and contains a mixture of alpha and beta amino acid residues in the pattern α3/β1. Methods using the peptides of the present invention for inhibiting Ras signaling in a cell, promoting cell death, and treating, preventing, and/or diagnosing a cellular proliferative disorder, differentiative disorder, and/or neoplastic condition in a subject in need thereof are also disclosed.

This application claims the benefit of U.S. Provisional Patent Application Ser. No. 61/943,339, entitled “Alpha3Beta Hydrogen Bond Surrogate Macrocycles as Modulators of Ras,” filed on Feb. 22, 2014, and U.S. Provisional Patent Application Ser. No. 61/943,363, entitled “Alpha3Beta Hydrogen Bond Surrogate Macrocycles as Modulators of Ras,” filed on Feb. 22, 2014, each of which is incorporated herein by reference in its entirety.

This invention was made with U.S. government support under grant numbers R01GM073943 and R01GM078266 awarded by the National Institutes of Health. The government has certain rights in this invention.

### FIELD OF THE INVENTION

This invention is directed generally to peptides having a stable, internally constrained protein secondary structure, where the peptide contains a hydrogen bond surrogate in the internal constraint, and at least one beta amino acid.

### BACKGROUND OF THE INVENTION

Aberrant receptor tyrosine kinase (RTK) signaling is a major underlying cause of various developmental disorders and hyperproliferative diseases (Blume-Jensen et al., “Oncogenic Kinase Signalling,” Nature 411:355 (2001)). A primary transduction mechanism by which RTK signals are propagated to intracellular pathways involves the ligand-dependent activation of the small guanine nucleotide binding protein Ras (FIG. 1) (Buday et al., “Many Faces of Ras Activation,” Biochim. Biophys. Acta 1786:178 (2008)). Accordingly, design of Ras signaling pathway inhibitors has been an active area of research for anticancer therapy (Downward et al., “Targeting Ras Signalling Pathways in Cancer Therapy,” Nat. Rev. Cancer 3:11 (2003)). The rate-limiting step in Ras activation process is the conversion of Ras-GDP to Ras-GTP through an exchange reaction that is catalyzed by the Ras specific guanine nucleotide exchange factor Sos (FIG. 2). The highly conserved catalytic domain (Rem+cdc25) of Sos interacts with Ras at a helical hairpin composed of the α-H and α-I helices (FIG. 3). The helical hairpin may be capable of nucleotide dissociation from Ras and subsequent down-regulation of the Ras pathway (Sacco et al., “The Isolated Catalytic Hairpin of the Ras-Specific Guanine Nucleotide Exchange Factor Cdc25(Mm) Retains Nucleotide Dissociation Activity but Has Impaired Nucleotide Exchange Activity,” FEBS Lett. 579:6851 (2005)). The high resolution structures of this complex suggest that the α-H helix is the only portion of the helical hairpin that makes direct contact with Ras, while the α-I helix may only serve to stabilize the α-H conformation (Boriack-Sjodin et al., “The Structural Basis of the Activation of Ras by Sos,” Nature 394:337 (1998)).

Inhibitors of the Ras-Sos interactions would be valuable as tools to dissect this complex signaling pathway and as leads for anticancer drug design. Therefore, there remains a need for methods and compositions for treating developmental disorders and hyperproliferative diseases by inhibiting undesirable activities associated with Ras proteins, for example by inhibition of the Ras/Sos complex.

The present invention is directed to overcoming these and other deficiencies in the art.

### SUMMARY OF THE INVENTION

A first aspect of the present invention relates to a peptide having a stable, internally-constrained HBS α-helix, where the peptide mimics at least a portion of the α-H helix of the Sos protein and contains a mixture of alpha and beta amino acid residues in the pattern α3/β1.

A second aspect of the present invention relates to a pharmaceutical composition comprising a peptide of the present invention and a pharmaceutically acceptable vehicle.

A third aspect of the present invention relates to a method of inhibiting Ras signaling in a cell, comprising contacting the cell with a peptide of the present invention. under conditions effective to inhibit Ras signaling in the cell

A fourth aspect of the present invention relates to a method of promoting cell death, comprising contacting the cell with a peptide of the present invention under conditions effective for the peptide to promote cell death.

A fifth aspect of the present invention relates to a method of treating, preventing, and/or diagnosing a cellular proliferative disorder, differentiative disorder, and/or neoplastic condition in a subject in need thereof, comprising administering to the subject a peptide of the present invention.

RAS gene mutations are associated with roughly 30% of human cancers, and are established drivers of the tumorigenic process. While the function of wild-type Ras is governed by its interaction with SOS, oncogenic Ras had been considered to acquire functional signaling autonomy. Accumulating evidence challenges this viewpoint and assigns a direct role for Sos-catalyzed activation of wild type Ras in oncogenic Ras-driven cancers. Here we show that potent helical mimics of Sos that inhibits Ras activation can decrease the viability of mutant Ras cancer cells. It has also been found that judicious insertion of β³-amino acid residues in Sos α-H HBS peptides considerably improves their binding affinity.

### BRIEF DESCRIPTION OF THE DRAWINGS

FIG. 1 is a schematic illustration of the Ras signaling pathway.

FIG. 2 is a schematic illustration showing the relationship between Ras and Sos. The activity of Ras is facilitated by the specific guanine nucleotide exchange factor Sos. Activated Ras controls a multitude of signaling transduction pathways.

FIG. 3 is a model showing the key α-helical interface between Ras and Sos (Protein Data Bank Accession No. 1BKD).

FIG. 4 shows depictions of α-HBS_{SOS}, α₃β-HBS_{SOS}, α₃β-HBS_{MUT}, and α₃β-UNC_{SOS}.

FIG. 5 is a schematic showing the synthesis of HBS α3β peptides.

FIG. 6 is the circular dichroism spectra of α3β-peptides.

FIG. 7 is a graph of the initial rates of proteolytic cleavage of α3β peptides by trypsin.

FIG. 8 is a graph of the binding affinities of the fluorescein-labeled SOS peptides interrogated in a cell-free pull-down assay with HRas⁽¹⁻¹⁶⁶⁾.

FIG. 9 shows depictions of fluorescein labeled α₃β-peptides.

FIG. 10 is a graph of the rates of nucleotide exchange from Ras in the presence or absence of Sos mimics.

FIGS. 11A-B are western blots (FIG. 11A) and a graph (FIG. 11B) showing that WT HRas and NRas activation is abrogated, and oncogenic KRas GTP-loading is significantly reduced, upon treatment of MIA PaCa-2 cells with α₃β-HBS_{SOS}.

FIGS. 12A-B are graphs showing that α₃β-HBS_{SOS} reduces the viability of various cancer cell lines in a dose dependent manner.

FIGS. 13A-D are images (FIGS. 13A-B) and graphs (Figures C-D) showing that the macropinocytic index predicts the sensitivity of cancer cells to α₃β-HBS_{SOS}.

FIGS. 14A-B show that Ras mutational status underlies the mechanism of α₃β-HBS_{SOS} induced cytotoxicity.

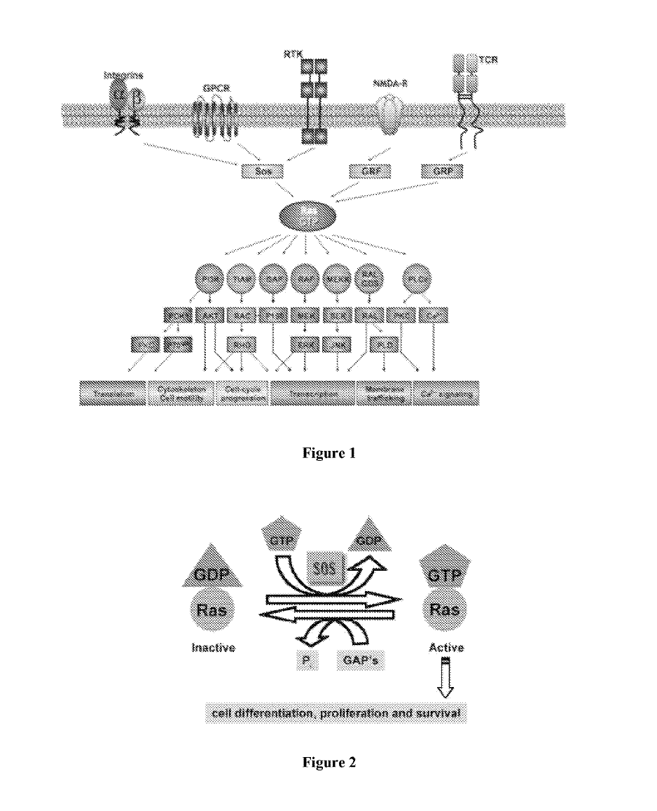

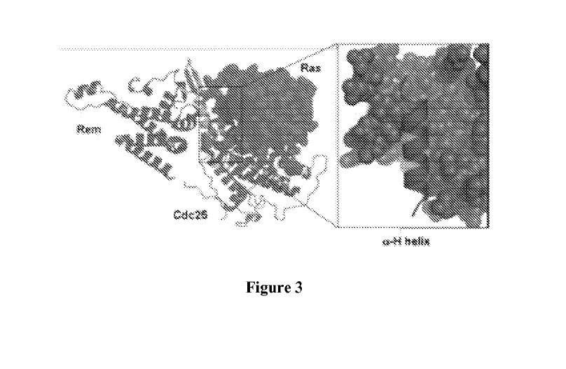

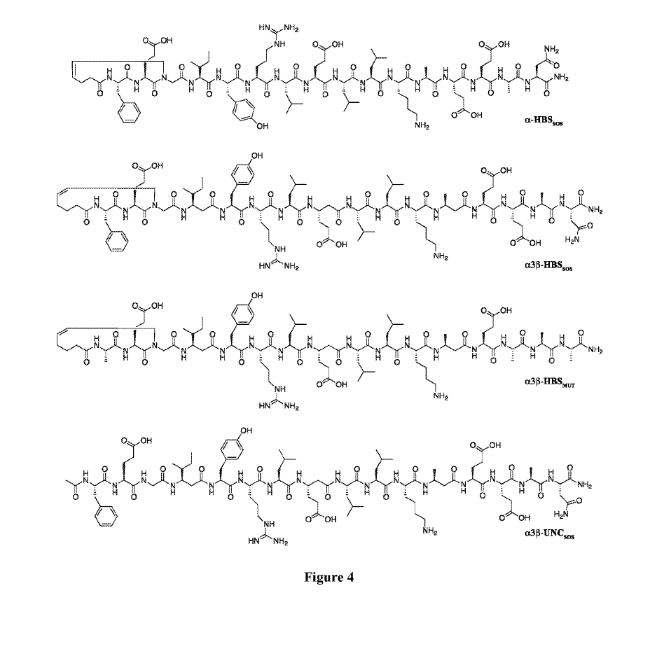

### DETAILED DESCRIPTION OF THE INVENTION

The Sos protein comprises two α-helical structural domains, including an N-terminal domain (amino acids 568-741, encompassing α-helices α1 through α6) and a C-terminal domain (amino acids 752-1044, encompassing α-helices αA through αK). The C-terminal domain of the Sos protein is involved primarily in interaction with Ras. In particular, helix αH plays an important role in the nucleotide exchange mechanism. The present invention relates to hydrogen bond surrogate (“HBS”) peptides containing a combination of alpha and beta amino acid residues in a α3/β1 pattern (“α₃β HBS”) capable of disrupting the Ras signaling pathway. These α₃β HBS helices can potentially function as in vivo inhibitors of Ras/Sos interaction.

One aspect of the present invention relates to a peptide having a stable, internally-constrained HBS α-helix, where the peptide mimics at least a portion of the α-H helix of the Sos protein and contains a mixture of alpha and beta amino acid residues in the pattern α3/β1.

The term “mimic” refers to the ability of a composition of the invention to effect a similar activity as a natural protein (e.g., Sos). A “mimic” encompasses both functional and structural mimics of such proteins. For example, the mimic is a protein which shares a certain percent homology (e.g., 60%, 70%, 80%, 85%, 90%, or 95% homology) with the target protein. Alternatively, the mimic is derived from a different sequence that nevertheless is capable of interacting with Ras in a functionally similar manner, for example by interacting with the same active site.

In a preferred embodiment, the peptide comprises a sequence of formula (X/Z)-AA¹-AA²-AA³*-AA⁴-AA⁵-AA⁶-AA⁷-AA⁸-AA⁹-AA¹⁰-AA¹¹-AA¹²-AA¹³-AA¹⁴-AA¹⁵-AA¹⁶, wherein X is 4-pentenoic acid; Z is 5-hexenoic acid; AA¹-AA¹⁶ are each independently an alpha or beta amino acid residue, wherein AA¹ is Phe; AA² is any amino acid residue (e.g., Glu or Asp); AA³ is Gly or Ala; AA⁴ is any amino acid residue; AA⁵ is any charged (preferably positively) and/or aromatic amino acid residue (e.g., Tyr, Phe, Trp, Arg, or Lys); AA⁶ is any amino acid residue (e.g., Arg or Lys); AA⁷ is an amino acid residue that is hydrophobic and aliphatic or able to form a hydrogen bond (e.g., Leu, Ile, Val, Thr, or Ser); AA⁸ is any amino acid residue (e.g., Glu, Asp, Gln, Asn, Arg, or Lys); AA⁹ is any amino acid residue; AA¹⁰ is Leu or any charged amino acid residue (e.g., Leu, Arg, Lys, His, Glu, or Asp); AA¹¹ is any amino acid residue (e.g., Lys or Arg); AA¹² is any amino acid residue; AA¹³ is any charged amino acid residue (e.g., Glu, Asp, Lys, or Arg); AA¹⁴ is any amino acid residue (e.g., Glu); AA¹⁵ is any amino acid residue (e.g., Ala or Gly); AA¹⁶ is Asn; and * denotes the placement of the internal constraint (i.e., between (X/Z) and R³). Suitable sequences include, e.g., (X/Z)FEG*iYRLeLLKaEEAN, (X/Z)FEg*IYRIELLkAEEaN, XFeG*IYrLELlKAEeAN, XfEG*IyRLEILKAeEAN, XFEG*iYRLeLLKaEEAN, ZFEG*iYRLeLLKaEEAn, ZFEG*iYRTeLLKaEEAN, ZFEG*iYRLqLLKaEEAN, ZFEg*IYRlELLkAEEaN, XFEg*IYRlELLkAEEaN, ZFEg*IYRtELLkAEEaN, ZFEg*IYRlQLLkAEEaN, XFeG*IYrTELlKAEeAN, XFeG*IYrLQLlKAEeAN, XfEG*IyRTElLKAeEAN, and XfEG*IyRLQlLKAeEAN, wherein lower case denotes beta amino acid residues.

In a preferred embodiment, the peptide is a peptide of Formula I:

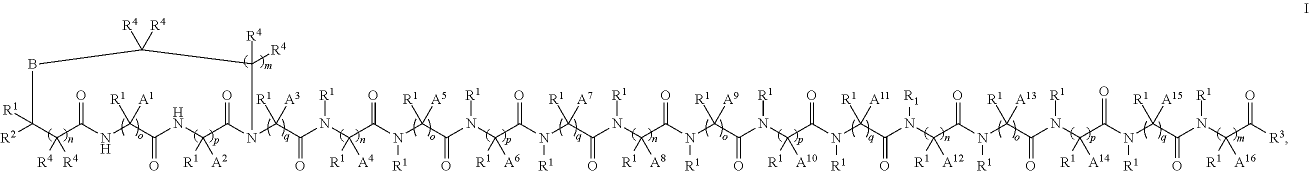

wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
m is one or two;
each n is the same and is one or two;
each o is the same and is one or two;
each p is the same and is one or two; and
each q is the same and is one or two;
wherein at least one of the following conditions is met
   (i) n, o, and p are one and q is two;
   (ii) n, o, and q are one and p is two;
   (iii) n, p, and q are one and o is two;
   (iv) o, p, and q are one and n is two.

Unless otherwise specified, amino acid side chains according to this and all aspects of the present invention can be any amino acid side chain from natural or nonnatural amino acids, including from alpha amino acids, beta amino acids, gamma amino acids, L-amino acids, and D-amino acids. In the sequences described herein, lower case letters denote beta amino acid residues.

As used herein, the term “alkyl” means an aliphatic hydrocarbon group which may be straight or branched having about 1 to about 6 carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 6 carbon atoms in the chain. Preferred alkenyl groups have 2 to about 4 carbon atoms in the chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having about 2 to about 6 carbon atoms in the chain. Preferred alkynyl groups have 2 to about 4 carbon atoms in the chain. Exemplary alkynyl groups include ethynyl, propynyl, n-butynyl, 2-butynyl, 3-methylbutynyl, and n-pentynyl.

As used herein, the term “cycloalkyl” refers to a non-aromatic saturated or unsaturated mono- or polycyclic ring system which may contain 3 to 6 carbon atoms, and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

As used herein, the term “heterocyclyl” refers to a stable 3- to 18-membered ring system that consists of carbon atoms and from one to five heteroatoms selected from the group consisting of nitrogen, oxygen, and sulfur. The heterocyclyl may be a monocyclic or a polycyclic ring system, which may include fused, bridged, or spiro ring systems; and the nitrogen, carbon, or sulfur atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like.

As used herein, the term “aryl” refers to an aromatic monocyclic or polycyclic ring system containing from 6 to 19 carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein, “heteroaryl” refers to an aromatic ring system that consists of carbon atoms and from one to five heteroatoms selected from the group consisting of nitrogen, oxygen, and sulfur. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl.

The term “arylalkyl” refers to a moiety of the formula —R^{a}R^{b} where R^{a} is an alkyl or cycloalkyl as defined above and R^{b} is an aryl or heteroaryl as defined above.

As used herein, the term “acyl” means a moiety of formula R-carbonyl, where R is an alkyl, cycloalkyl, aryl, or heteroaryl as defined above. Exemplary acyl groups include formyl, acetyl, propanoyl, benzoyl, and propenoyl.

An amino acid according to this and all aspects of the present invention can be any natural or non-natural amino acid.

A “peptide” as used herein is any oligomer of two or more natural or non-natural amino acids, including alpha amino acids, beta amino acids, gamma amino acids, L-amino acids, D˜amino acids, and combinations thereof. In preferred embodiments, the peptide is ˜5 to ˜30 (e.g., ˜5 to ˜10, ˜5 to ˜17, ˜10 to ˜17, ˜10 to ˜30, or ˜18 to ˜30) amino acids in length. Typically, the peptide is 10-17 amino acids in length. In a preferred embodiment, the peptide contains a mixture of alpha and beta amino acids in the pattern α3/β1.

A “tag” as used herein includes any labeling moiety that facilitates the detection, quantitation, separation, and/or purification of the compounds of the present invention. Suitable tags include purification tags, radioactive or fluorescent labels, and enzymatic tags.

Purification tags, such as poly-histidine (His₆), a glutathione-S-transferase (GST-), or maltose-binding protein (MBP-), can assist in compound purification or separation but can later be removed, i.e., cleaved from the compound following recovery. Protease-specific cleavage sites can be used to facilitate the removal of the purification tag. The desired product can be purified further to remove the cleaved purification tags.

Other suitable tags include radioactive labels, such as, ¹²⁵I, ¹³¹I, ¹¹¹In, or ⁹⁹TC. Methods of radiolabeling compounds are known in the art and described in U.S. Pat. No. 5,830,431 to Srinivasan et al., which is hereby incorporated by reference in its entirety. Radioactivity is detected and quantified using a scintillation counter or autoradiography. Alternatively, the compound can be conjugated to a fluorescent tag. Suitable fluorescent tags include, without limitation, chelates (europium chelates), fluorescein and its derivatives, rhodamine and its derivatives, dansyl, Lissamine, phycoerythrin, and Texas Red. The fluorescent labels can be conjugated to the compounds using techniques disclosed in CURRENT PROTOCOLS IN IMMUNOLOGY (Coligen et al. eds., 1991), which is hereby incorporated by reference in its entirety. Fluorescence can be detected and quantified using a fluorometer.

Enzymatic tags generally catalyze a chemical alteration of a chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Examples of suitable enzymatic tags include luciferases (e.g., firefly luciferase and bacterial luciferase; see e.g., U.S. Pat. No. 4,737,456 to Weng et al., which is hereby incorporated by reference in its entirety), luciferin, 2,3-dihydrophthalazinediones, malate dehydrogenase, urease, peroxidases (e.g., horseradish peroxidase), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to proteins and peptides are described in O'Sullivan et al., Methods for the Preparation of Enzyme-Antibody Conjugates for Use in Enzyme Immunoassay, in METHODS IN ENZYMOLOGY 147-66 (Langone et al. eds., 1981), which is hereby incorporated by reference in its entirety.

A targeting moiety according to the present invention functions to (i) promote the cellular uptake of the compound, (ii) target the compound to a particular cell or tissue type (e.g., signaling peptide sequence), or (iii) target the compound to a specific sub-cellular localization after cellular uptake (e.g., transport peptide sequence).

To promote the cellular uptake of a compound of the present invention, the targeting moiety may be a cell penetrating peptide (CPP). CPPs translocate across the plasma membrane of eukaryotic cells by a seemingly energy-independent pathway and have been used successfully for intracellular delivery of macromolecules, including antibodies, peptides, proteins, and nucleic acids, with molecular weights several times greater than their own. Several commonly used CPPs, including polyarginines, transportant, protamine, maurocalcine, and M918, are suitable targeting moieties for use in the present invention and are well known in the art (see Stewart et al., “Cell-Penetrating Peptides as Delivery Vehicles for Biology and Medicine,” Organic Biomolecular Chem. 6:2242-2255 (2008), which is hereby incorporated by reference in its entirety). Additionally, methods of making CPP are described in U.S. Patent Application Publication No. 20080234183 to Hallbrink et al., which is hereby incorporated by reference in its entirety.

Another suitable targeting moiety useful for enhancing the cellular uptake of a compound is an “importation competent” signal peptide as disclosed by U.S. Pat. No. 6,043,339 to Lin et al., which is hereby incorporated by reference in its entirety. An importation competent signal peptide is generally about 10 to about 50 amino acid residues in length-typically hydrophobic residues—that render the compound capable of penetrating through the cell membrane from outside the cell to the interior of the cell. An exemplary importation competent signal peptide includes the signal peptide from Kaposi fibroblast growth factor (see U.S. Pat. No. 6,043,339 to Lin et al., which is hereby incorporated by reference in its entirety). Other suitable peptide sequences can be selected from the SIGPEP database (see von Heijne G., “SIGPEP: A Sequence Database for Secretory Signal Peptides,” Protein Seq. Data Anal. 1(1):41-42 (1987), which is hereby incorporated by reference in its entirety).

Another suitable targeting moiety is a signal peptide sequence capable of targeting the compounds of the present invention to a particular tissue or cell type. The signaling peptide can include at least a portion of a ligand binding protein. Suitable ligand binding proteins include high-affinity antibody fragments (e.g., Fab, Fab′ and F(ab′)₂, single-chain Fv antibody fragments), nanobodies or nanobody fragments, fluorobodies, or aptamers. Other ligand binding proteins include biotin-binding proteins, lipid-binding proteins, periplasmic binding proteins, lectins, serum albumins, enzymes, phosphate and sulfate binding proteins, immunophilins, metallothionein, or various other receptor proteins. For cell specific targeting, the signaling peptide is preferably a ligand binding domain of a cell specific membrane receptor. Thus, when the modified compound is delivered intravenously or otherwise introduced into blood or lymph, the compound will adsorb to the targeted cell, and the targeted cell will internalize the compound. For example, if the target cell is a cancer cell, the compound may be conjugated to an anti-C3B(I) antibody as disclosed by U.S. Pat. No. 6,572,856 to Taylor et al., which is hereby incorporated by reference in its entirety. Alternatively, the compound may be conjugated to an alphafeto protein receptor as disclosed by U.S. Pat. No. 6,514,685 to Moro, which is hereby incorporated by reference in its entirety, or to a monoclonal GAH antibody as disclosed by U.S. Pat. No. 5,837,845 to Hosokawa, which is hereby incorporated by reference in its entirety. For targeting a compound to a cardiac cell, the compound may be conjugated to an antibody recognizing elastin microfibril interfacer (EMILIN2) (Van Hoof et al., “Identification of Cell Surface for Antibody-Based Selection of Human Embryonic Stem Cell-Derived Cardiomyocytes,” J Proteom Res 9:1610-18 (2010), which is hereby incorporated by reference in its entirety), cardiac troponin I, connexin-43, or any cardiac cell-surface membrane receptor that is known in the art. For targeting a compound to a hepatic cell, the signaling peptide may include a ligand domain specific to the hepatocyte-specific asialoglycoprotein receptor. Methods of preparing such chimeric proteins and peptides are described in U.S. Pat. No. 5,817,789 to Heartlein et al., which is hereby incorporated by reference in its entirety.

Another suitable targeting moiety is a transport peptide that directs intracellular compartmentalization of the compound once it is internalized by a target cell or tissue. For transport to the endoplasmic reticulum (ER), for example, the compound can be conjugated to an ER transport peptide sequence. A number of such signal peptides are known in the art, including the signal peptide MMSFVSLLLVGILFYATEAEQLTKCEVFQ (SEQ ID NO: 1). Other suitable ER signal peptides include the N-terminus endoplasmic reticulum targeting sequence of the enzyme 17@-hydroxysteroid dehydrogenase type 11 (Horiguchi et al., “Identification and Characterization of the ER/Lipid Droplet-Targeting Sequence in 17@-hydroxysteroid Dehydrogenase Type 11,” Arch. Biochem. Biophys. 479(2):121-30 (2008), which is hereby incorporated by reference in its entirety), or any of the ER signaling peptides (including the nucleic acid sequences encoding the ER signal peptides) disclosed in U.S. Patent Application Publication No. 20080250515 to Reed et al., which is hereby incorporated by reference in its entirety. Additionally, the compound of the present invention can contain an ER retention signal, such as the retention signal KEDL (SEQ ID NO: 2). Methods of modifying the compounds of the present invention to incorporate transport peptides for localization of the compounds to the ER can be carried out as described in U.S. Patent Application Publication No. 20080250515 to Reed et al., which is hereby incorporated by reference in its entirety.

For transport to the nucleus, the compounds of the present invention can include a nuclear localization transport signal. Suitable nuclear transport peptide sequences are known in the art, including the nuclear transport peptide PPKKKRKV (SEQ ID NO: 3). Other nuclear localization transport signals include, for example, the nuclear localization sequence of acidic fibroblast growth factor and the nuclear localization sequence of the transcription factor NF-KB p50 as disclosed by U.S. Pat. No. 6,043,339 to Lin et al., which is hereby incorporated by reference in its entirety. Other nuclear localization peptide sequences known in the art are also suitable for use in the compounds of the present invention.

Suitable transport peptide sequences for targeting to the mitochondria include MLSLRQSIRFFKPATRTLCSSRYLL (SEQ ID NO: 4). Other suitable transport peptide sequences suitable for selectively targeting the compounds of the present invention to the mitochondria are disclosed in U.S. Patent Application Publication No. 20070161544 to Wipf, which is hereby incorporated by reference in its entirety.

In at least one embodiment, n, o, and p are one and q is two. In at least one embodiment, p is two. In at least one embodiment, n, o, and q are one and p is two. In at least one embodiment, n, p, and q are one and o is two. In at least one embodiment, o, p, and q are one and n is two.

The peptides according to all aspects of the present invention can be prepared using the methods disclosed in U.S. Pat. No. 7,202,332 to Arora & Chapman (when B is carbon) and U.S. Provisional Patent Application No. 61/529,414 to Arora & Mahon (when B is S, O, or N), each of which is hereby incorporated by reference in its entirety), but using beta amino acids in place of alpha amino acids, as appropriate. See, for example, Examples 1-2, infra.

Suitable peptides according to this and all aspects of the present invention include, for example, (X/Z)FEG*iYRLeLLKaEEAN-NH₂, (X/Z)FEg*IYRlELLkAEEaN-NH₂, XFeG*IYrLELlKAEeAN-NH₂, XfEG*IyRLElLKAeEAN-NH₂, XFEG*iYRLeLLKaEEAN-NH₂, ZFEG*iYRLeLLKaEEAn-NH₂, ZFEG*iYRTeLLKaEEAN-NH₂, ZFEG*iYRLqLLKaEEAN-NH₂, ZFEg*IYRlELLkAEEaN-NH₂, XFEg*IYRlELLkAEEaN-NH₂, ZFEg*IYRtELLkAEEaN-NH₂, ZFEg*IYRlQLLkAEEaN-NH₂, XFeG*IYrTELlKAEeAN-NH₂, XFeG*IYrLQLlKAEeAN-NH₂, XfEG*IyRTElLKAeEAN-NH₂, and XfEG*IyRLQlLKAeEAN-NH₂.

Another aspect of the present invention relates to a pharmaceutical composition comprising a peptide of the present invention and a pharmaceutically acceptable vehicle.

Yet another aspect of the present invention relates to a method of inhibiting Ras signaling in a cell. This method involves contacting the cell with a peptide of the present invention under conditions effective to inhibit Ras signaling in the cell.

Contacting a cell with a peptide according to this and all aspects of the present invention may be carried out in vitro or in vivo.

When contacting is carried out in vivo, contacting may comprise administering to a subject a compound that includes the peptide of the present invention. As will be apparent to one of ordinary skill in the art, administering may be carried out using generally known methods.

Administration can be accomplished either via systemic administration to the subject or via targeted administration to affected cells. Exemplary routes of administration include, without limitation, by intratracheal inoculation, aspiration, airway instillation, aerosolization, nebulization, intranasal instillation, oral or nasogastric instillation, intraperitoneal injection, intravascular injection, topically, transdermally, parenterally, subcutaneously, intravenous injection, intra-arterial injection (such as via the pulmonary artery), intramuscular injection, intrapleural instillation, intraventricularly, intralesionally, by application to mucous membranes (such as that of the nose, throat, bronchial tubes, genitals, and/or anus), or implantation of a sustained release vehicle.

Typically, the peptide of the present invention will be administered to a mammal as a pharmaceutical formulation that includes the therapeutic agent and any pharmaceutically acceptable adjuvants, carriers, excipients, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of therapeutic agent together with the adjuvants, carriers, and/or excipients. The amount of active compound in such therapeutically useful compositions is such that a suitable dosage unit will be obtained

The agents may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0. 1% of the agent. The percentage of the agent in these compositions may, of course, be varied and may conveniently be between about 2% to about 60% of the weight of the unit. The amount of the agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient(s), sucrose as a sweetening agent, methyl and propylparabens as preservatives, a dye, and flavoring.

The agents may also be administered parenterally. Solutions or suspensions of the agent can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents according to this aspect of the present invention may also be administered directly to the airways in the form of an aerosol. For use as aerosols, the compounds of the present invention in solution or suspension may be packaged in a pressurized aerosol container together with suitable propellents, for example, hydrocarbon propellants like propane, butane, or isobutane, with conventional adjuvants. The materials of the present invention also may be administered in a non-pressurized form such as in a nebulizer or atomizer.

The agents of the present invention may be administered directly to a targeted tissue, e.g., tissue that is susceptible to the condition to be treated. Additionally and/or alternatively, the agent may be administered to a non-targeted area along with one or more agents that facilitate migration of the agent to (and/or uptake by) a targeted tissue, organ, or cell. As will be apparent to one of ordinary skill in the art, the therapeutic agent itself may be modified to facilitate its transport to (and uptake by) the desired tissue, organ, or cell.

Exemplary delivery devices include, without 1 imitation, nebulizers, atomizers, liposomes, transdermal patches, implants, implantable or injectable protein depot compositions, and syringes. Other delivery systems which are known to those of skill in the art can also be employed to achieve the desired delivery of the therapeutic agent to the desired organ, tissue, or cells in vivo to effect this aspect of the present invention.

Any suitable approach for delivery of the agents can be utilized to practice this aspect of the present invention. Typically, the agent will be administered to a patient in a vehicle that delivers the agent(s) to the target cell, tissue, or organ.

One approach for delivering agents into cells involves the use of liposomes. Basically, this involves providing a liposome which includes agent(s) to be delivered, and then contacting the target cell, tissue, or organ with the liposomes under conditions effective for delivery of the agent into the cell, tissue, or organ.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang & Huang, “pH-Sensitive Immunoliposomes Mediate Target-Cell-Specific Delivery and Controlled Expression of a Foreign Gene in Mouse,” Proc. Nat'l Acad. Sci. USA 84:7851-55 (1987), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., each of which is hereby incorporated by reference in its entirety.

These liposomes can be produced such that they contain, in addition to the therapeutic agents of the present invention, other therapeutic agents, such as anti-inflammatory agents, which would then be released at the target site (e.g., Wolff et al., “The Use of Monoclonal Anti-ThyI IgG1 for the Targeting of Liposomes to AKR-A Cells in Vitro and in Vivo,” Biochim. Biophys. Acta 802:259-73 (1984), which is hereby incorporated by reference in its entirety).

An alternative approach for delivery of proteins or polypeptide agents (e.g., peptides of the present invention) involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptide agents involves preparation of chimeric proteins according to U.S. Pat. No. 5,817,789 to Heartlein et al., which is hereby incorporated by reference in its entirety. The chimeric protein can include a ligand domain and the polypeptide agent (e.g., the artificial α-helix of the present invention). The ligand domain is specific for receptors located on a target cell. Thus, when the chimeric protein is delivered intravenously or otherwise introduced into blood or lymph, the chimeric protein will adsorb to the targeted cell, and the targeted cell will internalize the chimeric protein.

Administration can be carried out as frequently as required and for a duration that is suitable to provide effective treatment. For example, administration can be carried out with a single sustained-release dosage formulation or with multiple daily doses.

The amount to be administered will, of course, vary depending upon the treatment regimen.

Generally, an agent is administered to achieve an amount effective for an improvement in the state of the patient (i.e., a therapeutically effective amount). Thus, in the case of cancer, a therapeutically effective amount can be an amount which is capable of at least partially decreasing the size of a tumor, decreasing the number of cancerous cells in the body, or slowing the increase in the number of cancer cells in the body. The dose required to obtain an effective amount may vary depending on the agent, formulation, cancer, and individual to whom the agent is administered.

Determination of effective amounts may also involve in vitro assays in which varying doses of agent are administered to cells in culture and the concentration of agent effective for inhibiting growth of cancer cells is determined in order to calculate the concentration required in vivo. Effective amounts may also be based on in vivo animal studies. A therapeutically effective amount can be determined empirically by those of skill in the art.

When using this method to treat a subject, the above-mentioned modes and forms of administering are used to contact the cell with the one or more peptides of the present invention.

Yet another aspect of the present invention relates to a method for promoting cell death. This method involves contacting a cell with a peptide of the present invention under conditions effective for the peptide to promote cell death. Contacting may be carried out as described above.

Another aspect of the present invention relates to a method of using the peptides of the present invention to treat, prevent, and/or diagnose cancers and neoplastic conditions. As used herein, the terms “cancer”, “hyperproliferative”, and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues, or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the compounds are novel therapeutic agents for controlling breast cancer, ovarian cancer, colon cancer, pancreatic cancer, bladder cancer, lung cancer, metastasis of such cancers, and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AL) and chronic myelogenous leukemia (CL) (reviewed in Vaickus et al., “Immune Markers in Hematologic Malignancies,” Crit. Rev. Oncol. Hemotol. 11:267-97 (1991), which is hereby incorporated by reference in its entirety). Lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL), and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease, and Reed-Stemberg disease.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic rumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

In some embodiments, the peptides of the present invention are used to treat a cancer mediated by a mutated Ras protein. Cancers known to frequently involve such mutations include, but are not limited to, non-small-cell lung cancer (adenocarcinoma), colorectal cancer, pancreatic cancer, thyroid cancers (e.g., follicular, undifferentiated papillary or papillary), seminoma, melanoma, bladder cancer, liver cancer, kidney cancer, myelodysplastic syndrome, and acute myelogenous leukemia.

### Breast Cancer

In some embodiments, the invention provides methods of treating breast cancer by administering the peptides of the invention. Breast cancer includes invasive breast carcinomas, such as invasive ductal carcinoma, invasive lobular carcinoma, tubular carcinoma, invasive cribriform carcinoma, medullary carcinoma, mucinous carcinoma and other tumors with abundant mucin, cystadenocarcinoma, columnar cell mucinous carcinoma, signet ring cell carcinoma, neuroendocrine tumors (including solid neuroendocrine carcinoma, atypical carcinoid tumour, small cell/oat cell carcinoma, or large cell neuroendocrine carcinoma), invasive papillary carcinoma, invasive micropapillary carcinoma, apocrine carcinoma, metaplastic carcinomas, pure epithelial metaplastic carcinomas, mixed epithelial/mesenchymal metaplastic carcinomas, lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma or bilateral breast carcinoma; mesenchymal tumors such as haemangioma, angiomatosis, haemangiopericytoma, pseudoangiomatous stromal hyperplasia, myofibroblastoma, fibromatosis (aggressive), inflammatory myofibroblast tumour, lipoma, angiolipoma, granular cell tumour, neurofibroma, schwannoma, angiosarcoma, liposarcoma, rhabdomyosarcoma, osteosarcoma, leiomyoma, or leiomysarcoma; myoepithelial lesions such as myoepitheliosis, adenomyoepithelial adenosis, adenomyoepithelioma, or malignant myoepithelioma; fibroepithelial tumors such as fibroadenoma, phyllodes tumour, low grade periductal stromal sarcoma, or mammary hamartoma; and tumors of the nipple such as nipple adenoma, syringomatous adenoma, or Paget's disease of the nipple.

Treatment of breast cancer may be effected in conjunction with any additional therapy, such as a therapy that is part of the standard of care. A surgical technique such as lumpectomy or mastectomy may be performed prior to, during, or following treatment with the peptides of the present invention. Alternatively, radiation therapy may be used for the treatment of breast cancer in conjunction with the peptides of the present invention. In other cases, the peptides of the present invention are administered in combination with a second therapeutic agent. Such an agent may be a chemotherapeutic agent such as an individual drug or combination of drugs and therapies. For example, the chemotherapeutic agent can be an adjuvant chemotherapeutic treatment such as CMF (cyclophosphamide, methotrexate, and 5-fluorouracil); FAC or CAF (5-fluorouracil, doxorubicin, cyclophosphamide); AC or CA (doxorubicin and cyclophosphamide); AC-Taxol (AC followed by paclitaxel); TAC (docetaxel, doxorubicin, and cyclophosphamide); FEC (5-fluorouracil, epirubicin, and cyclophosphamide); FECD (FEC followed by docetaxel); TC (docetaxel and cyclophosphamide). In addition to chemotherapy, trastuzumab may also be added to the regimen depending on the tumor characteristics (i.e., HER2/neu status) and risk of relapse. Hormonal therapy may also be appropriate before, during or following chemotherapeutic treatment. For example, tamoxifen may be administered or a compound in the category of aromatase inhibitors including, but not limited to aminogluthetimide, anastrozole, exemestane, formestane, letrozole, or vorozole. In other embodiments, an antiangiogenic agent may be used in combination therapy for the treatment of breast cancer. The antiangiogenic agent may be an anti-VEGF agent including, but not limited to bevacizumab.

### Ovarian Cancer

In some embodiments, the peptides of the present invention may be used to treat ovarian cancer. Ovarian cancers include ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Rukenberg tumors.

The peptides of the present invention may be administered in conjunction with a second therapy such as a therapy that is part of the standard of care. Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpingectomy.

Anti-cancer drugs that may be used include cyclophosphamide, etoposide, altretamine, and ifosfamide. Hormone therapy with the drug tamoxifen may be used to shrink ovarian tumors. Radiation therapy may be external beam radiation therapy and/or brachytherapy.

### Prostate Cancer

In some embodiments, the peptides of the present invention may be used to treat prostate cancer. Prostate cancers include adenocarcinomas and metastasized adenocarcinomas. The peptides of the present invention may be administered in conjunction with a second therapy such as a therapy that is part of the standard of care. Treatment for prostate cancer may involve surgery, radiation therapy, High Intensity Focused Ultrasound (HIFU), chemotherapy, cryosurgery, hormonal therapy, or any combination thereof. Surgery may involve prostatectomy, radical perineal prostatectomy, laparoscopic radical prostatectomy, transurethral resection of the prostate or orchiectomy. Radiation therapy may include external beam radiation therapy and/or brachytherapy. Hormonal therapy may include orchiectomy, administration of antiandrogens such as flutamide, bicalutamide, nilutamide, or cyproterone acetate; medications which inhibit the production of adrenal androgens such as DHEA, such as ketoconazole and aminoglutethimide; and GnRH antagonists or agonists such as Abarelix (Plenaxis®), Cetrorelix (Cetrotide®), Ganirelix (Antagon®), leuprolide, goserelin, triptorelin, or buserelin. Treatment with an anti-androgen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy includes, but is not limited to, administration of docetaxel, for example with a corticosteroid such as prednisone. Anti-cancer drugs such as doxorubicin, estramustine, etoposide, mitoxantrone, vinblastine, paclitaxel, carboplatin may also be administered to slow the growth of prostate cancer, reduce symptoms and improve the quality of life. Additional compounds such as bisphosphonate drugs may also be administered.

### Renal Cancer

In some embodiments, the peptides of the present invention may be used to treat renal cancer. Renal cancers include, but are not limited to, renal cell carcinomas, metastases from extra-renal primary neoplasms, renal lymphomas, squamous cell carcinomas, juxtaglomerular tumors (reninomas), transitional cell carcinomas, angiomyolipomas, oncocytomas and Wilm's tumors. The peptides of the present invention may be administered in conjunction with a second therapy such as a therapy that is part of the standard of care. Treatment for renal cancer may involve surgery, percutaneous therapies, radiation therapies, chemotherapy, vaccines, or other medication. Surgical techniques useful for treatment of renal cancer in combination with the peptides of the present invention include nephrectomy, which may include removal of the adrenal gland, retroperitoneal lymph nodes, and any other surrounding tissues affected by the invasion of the tumor. Percutaneous therapies include, for example, image-guided therapies which may involve imaging of a tumor followed by its targeted destruction by radiofrequency ablation or cryotherapy. In some cases, other chemotherapeutic or other medications useful in treating renal cancer may be alpha-interferon, interleukin-2, bevacizumab, sorafenib, sunitib, temsirolimus or other kinase inhibitors.

### Pancreatic Cancer

In some embodiments, the invention provides methods of treating pancreatic cancer by administering peptides of the present invention, such as a pancreatic cancer selected from the following: an epitheliod carcinoma in the pancreatic duct tissue and an adenocarcinoma in a pancreatic duct. The most common type of pancreatic cancer is an adenocarcinoma, which occurs in the lining of the pancreatic duct. Possible treatments available for pancreatic cancer include surgery, immunotherapy, radiation therapy, and chemotherapy. Possible surgical treatment options include a distal or total pancreatectomy and a pancreaticoduodenectomy (Whipple procedure). Radiation therapy may be an option for pancreatic cancer patients, specifically external beam radiation where radiation is focused on the tumor by a machine outside the body. Another option is intraoperative electron beam radiation administered during an operation.

Chemotherapy may also be used to treat pancreatic cancer patients. Suitable anti-cancer drugs include, but are not limited to, 5-fluorouracil (5-FU), mitomycin, ifosfamide, doxorubicin, streptozocin, chlorozotocin, and combinations thereof. The methods provided by the invention can provide a beneficial effect for pancreatic cancer patients, by administration of a polypeptide of the invention or a combination of administration of a compound and surgery, radiation therapy, or chemotherapy.

### Colon Cancer

In some embodiments, peptides of the present invention may be used for the treatment of colon cancer, including but not limited to non-neoplastic polyps, adenomas, familial syndromes, colorectal carcinogenesis, colorectal carcinoma, and carcinoid tumors. Possible treatments available for colon cancer that may be used in conjunction with the peptides of the present invention include surgery, chemotherapy, radiation therapy, or targeted drug therapy.

Radiation therapy may include external beam radiation therapy and/or brachytherapy.

Chemotherapy may be used to reduce the likelihood of metastasis developing, shrink tumor size, or slow tumor growth. Chemotherapy is often applied after surgery (adjuvant), before surgery (neo-adjuvant), or as the primary therapy if surgery is not indicated (palliative). For example, exemplary regimens for adjuvant chemotherapy involve the combination of infusional 5-fluorouracil, leucovorin, and oxaliplatin (FOLFOX). First line chemotherapy regimens may involve the combination of infusional 5-fluorouracil, leucovorin, and oxaliplatin (FOLFOX) with a targeted drug such as bevacizumab, cetuximab or panitumumab or infusional 5-fluorouracil, leucovorin, and irinotecan (FOLF1RI) with targeted drug such as bevacizumab, cetuximab or panitumumab. Other chemotherapeutic agents that may be useful in the treatment or prevention of colon cancer in combination with the peptides of the present invention are Bortezomib (Velcade®), Oblimersen (Genasense®, G3139), Gefitinib and Erlotinib (Tarceva®) and Topotecan (Hycamtin®).

### Lung Cancer

Some embodiments provide methods for the treatment of lung cancer using the peptides of the present invention. Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer, e.g., small cell lung carcinomas, accounts for 15-20% of lung cancers. Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy. Some anti-cancer drugs that may be used in chemotherapy to treat lung cancer in combination with the peptides of the present invention include cisplatin, carboplatin, paclitaxel, docetaxel, gemcitabine, vinorelbine, irinotecan, etoposide, vinblastine, gefitinib, ifosfamide, methotrexate, or a combination thereof. Photodynamic therapy (PDT) may be used to treat lung cancer patients. The methods described herein can provide a beneficial effect for lung cancer patients, by administration of a compound or a combination of administration of a compound and surgery, radiation therapy, chemotherapy, photodynamic therapy, or a combination thereof.

### Liver Disorders

Examples of cellular proliferative and/or differentiative disorders of the liver include, but are not limited to, nodular hyperplasias, adenomas, and malignant tumors, including primary carcinoma of the liver and metastatic tumors.

### Immunoproliferative Disorders

Immunoproliferative disorders (also known as “immunoproliferative diseases” or “immunoproliferative neoplasms”) are disorders of the immune system that are characterized by the abnormal proliferation of the primary cells of the immune system, which includes B cells, T cells, and Natural Killer (K) cells, or by the excessive production of immunoglobulins (also known as antibodies). Such disorders include the general categories of lymphoproliferative disorders, hypergammaglobulinemias, and paraproteinemias. Examples of such disorders include, but are not limited to, X-linked lymphoproliferative disorder, autosomal lymphoproliferative disorder, Hyper-Ig syndrome, heavy chain disease, and cryoglobulinemia. Other immunoproliferative disorders can be graft versus host disease (GVHD); psoriasis; immune disorders associated with graft transplantation rejection; T cell lymphoma; T cell acute lymphoblastic leukemia; testicular angiocentric T cell lymphoma; benign lymphocytic angiitis; and autoimmune diseases such as lupus erythematosus, Hashimoto's thyroiditis, primary myxedema, Graves' disease, pernicious anemia, autoimmune atrophic gastritis, Addison's disease, insulin dependent diabetes mellitis, good pasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulceratis colitis, Sjogren's syndrome, rheumatoid arthritis, polymyositis, scleroderma, and mixed connective tissue disease.

### Combination Treatments

In one embodiment, peptides of the present invention may be used for the treatment of cancer in conjunction with alkylating and alkylating-like agents. Such agents include, for example, nitrogen mustards such as chlorambucil, chlormethine, cyclophosphamide, ifosfamide, and melphalan; nitrosoureas such as carmustine, fotemustine, lomustine, and streptozocin; platinum therapeutic agents such as carboplatin, cisplatin, oxaliplatin, BBR3464, and satraplatin; or other agents, including but not limited to busulfan, dacarbazine, procarbazine, temozolomide, thiotepa, treosulfan, or uramustine.

In another embodiment, peptides of the present invention may be used in conjunction with an antineoplastic agent which is an antimetabolite. For example, such an antineoplastic agent may be a folic acid such as aminopterin, methotrexate, pemetrexed, or raltitrexed. Alternatively, the antineoplastic agent may be a purine, including but not limited to cladribine, clofarabine, fludarabine, mercaptopurine, pentostatin, thioguanine. In further embodiments, the antineoplastic agent may be a pyrimidine such as capecitabine, cytarabine, fluorouracil, floxuridine, and gemcitabine.

In still other embodiments, peptides of the present invention may be used in conjunction with an antineoplastic agent which is an spindle poison/mitotic inhibitor. Agents in this category include taxanes, for example docetaxel and paclitaxel; and vinca alkaloids such as vinblastine, vincristine, vindesine, and vinorelbine. In yet other embodiments, peptides of the present invention may be used in combination with an antineoplastic agent which is a cytotoxic/antitumor antibiotic from the anthracycline family such as daunorubicin, doxorubicin, epirubicin, idarubicin, mitoxantrone, pixantrone, or valrubicin; an antibiotic from the streptomyces family such as actinomycin, bleomycin, mitomycin, or plicamycin; or hydroxyurea. Alternatively, agents used for combination therapy may be topoisomerase inhibitors including, but not limited to camptothecin, topotecan, irinotecan, etoposide, or teniposide.

Alternatively, the antineoplastic agent may be an antibody or antibody-derived agent. For example, a receptor tyrosine kinase-targeted antibody such as cetuximab, panitumumab, or trastuzumab may be used. Alternatively, the antibody may be an anti-CD20 antibody such as rituximab or tositumomab, or any other suitable antibody including but not limited to alemtuzumab, bevacizumab, and gemtuzumab. In other embodiments, the antineoplastic agent is a photosensitizer such as aminolevulinic acid, methyl aminolevulinate, porfimer sodium, or verteporfin. In still other embodiments, the antineoplastic agent is a tyrosine kinase inhibitor such as dediranib, dasatinib, erlotinib, gefitinib, imatinib, lapatinib, nilotinib, sorafenib, sunitinib, or vandetanib. Other neoplastic agents suitable in the use of the invention include, for example, alitretinoin, tretinoin, altretamine, amsacrine, anagrelide, arsenic trioxide, asparaginase (pegaspargase), bexarotene, bortezomib, denileukin diftitox, estramustine, ixabepilone, masoprocol, or mitotane.

In other or further embodiments, the compounds described herein are used to treat, prevent or diagnose conditions characterized by overactive cell death or cellular death due to physiologic insult, etc. Some examples of conditions characterized by premature or unwanted cell death are or alternatively unwanted or excessive cellular proliferation include, but are not limited to hypocellular hypoplastic, acellular/aplastic, or hypercellular/hyperplastic conditions. Some examples include hematologic disorders including but not limited to fanconi anemia, aplastic anemia, thalassemia, congenital neutropenia, and myelodysplasia.

In other or further embodiments, the peptides of the present invention that act to decrease apoptosis are used to treat disorders associated with an undesirable level of cell death. Thus, in some embodiments, the anti-apoptotic peptides of the present invention are used to treat disorders such as those that lead to cell death associated with viral infection, e.g., infection associated with infection with human immunodeficiency virus (HIV). A wide variety of neurological diseases are characterized by the gradual loss of specific sets of neurons, and the anti-apoptotic peptides of the present invention are used, in some embodiments, in the treatment of these disorders. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

### Other Methods of Use

In other or further embodiments, the anti-apoptotic peptides of the present invention are used to treat all such disorders associated with undesirable cell death.

Some examples of immunologic disorders that are treated with the compounds described herein include but are not limited to organ transplant rejection, arthritis, lupus, 1BD, Crohn's disease, asthma, multiple sclerosis, diabetes, etc.

Some examples of neurologic disorders that are treated with the compounds described herein include but are not limited to Alzheimer's disease, Down's syndrome, Dutch type hereditary cerebral hemorrhage amyloidosis, reactive amyloidosis, familial amyloid nephropathy with urticaria and deafness, Muckle-Wells syndrome, idiopathic myeloma; macroglobulinemia-associated myeloma, familial amyloid polyneuropathy, familial amyloid cardiomyopathy, isolated cardiac amyloid, systemic senile amyloidosis, adult onset diabetes, Insulinoma, isolated atrial amyloid, medullary carcinoma of the thyroid, familial amyloidosis, hereditary cerebral hemorrhage with amyloidosis, familial amyloidotic polyneuropathy, scrapie, Creutzfeldt-Jacob disease, Gerstmann Straussler-Scheinker syndrome, bovine spongiform encephalitis, a prion-mediated disease, and Huntington's disease.

Some examples of endocrinologic disorders that are treated with the compounds described herein include but are not limited to diabetes, hypothyroidism, hypopituitarism, hypoparathyroidism, hypogonadism, etc.

Examples of cardiovascular disorders (e.g., inflammatory disorders) that are treated or prevented with the peptides of the present invention include, but are not limited to, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema, and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve, or other implantable devices. Preferred cardiovascular disorders include atherosclerosis, myocardial infarction, aneurism, and stroke.

The present invention may be further illustrated by reference to the following examples.

### EXAMPLES

### Example 1

### Peptide Synthesis

Peptides were synthesized on a CEM Liberty microwave peptide synthesizer using Fmoc solid-phase chemistry on Rink amide resin, and purified by reversed-phase HPLC (Patgiri et al., “Solid-Phase Synthesis of Short α-Helices Stabilized by the Hydrogen Bond Surrogate Approach,” Nat. Protoc. 5(10):1857-65 (2010), which is hereby incorporated by reference in its entirety). The identity and the purity of the peptides were confirmed by ESI-MS.

### Example 2

### Synthesis of HBS Helices

HBS-α₃β helices were synthesized as previously described (Patgiri et al., “Nucleation Effects in Peptide Foldamers,”. Am. Chem. Soc. 134(28):11495-502 (2012), which is hereby incorporated by reference in its entirety). Peptide sequences up to the i+5^{th} residue of the putative helix were synthesized using Fmoc solid-phase chemistry on Rink amide resin on a CEM Liberty Series microwave peptide synthesizer. The resin was treated with HOAt-activated o-Ns-N-allylglycine. The nitrobenzenesulfonyl group was removed with mercaptoethanol to obtain the N-allylpeptide, which was coupled sequentially with Fmoc-amino acids, followed by coupling of 4-pentenoic acid to afford the bis-olefin peptide (Patgiri et al., “Solid-Phase Synthesis of Short α-Helices Stabilized by the Hydrogen Bond Surrogate Approach,” Nat. Protoc. 5(10):1857-65 (2010), which is hereby incorporated by reference in its entirety). Ring-closing metathesis of the bis-olefin peptide was performed with Hoveyda-Grubbs II catalyst in dichloroethane under microwave irradiation as described (Patgiri et al., “Solid-Phase Synthesis of Short α-Helices Stabilized by the Hydrogen Bond Surrogate Approach,” Nat. Protoc. 5(10):1857-65 (2010); Chapman & Arora “Optimized Synthesis of Hydrogen-Bond Surrogate Helices: Surprising Effects of Microwave Heating on the Activity of Grubbs Catalysts,” Org. Lett. 8(25):5825-28 (2006), each of which is hereby incorporated by reference in its entirety). Metathesized peptides were cleaved from the resin using TFA/TIS/water (95:2.5:2.5), purified by reversed-phase HPLC (Cis column) and characterized by ESI-MS.

### Example 3

### Circular Dichroism Spectroscopy

CD spectra were recorded on AVIV 202SF CD spectrometer equipped with a temperature controller using 1 mm length cells and a scan speed of 0.5 nm/sec. The spectra were averaged over 10 scans with the baseline subtracted from analogous conditions as that for the samples. The samples were prepared in Tris buffer (15 mM NaCl, 1 mM Tris, pH 6.8), containing 10% trifluoroethanol, with the final peptide concentration of 90 μM. The concentrations of peptides were determined by the UV absorption of the tyrosine residue at 280 nm. The relative helix content of the peptides was determined from the mean residue CD at 205 nm, [θ]₂₂₂ (deg cm² dmol⁻¹) corrected for the number of amino acids (Wang et al., “Evaluation of Biologically Relevant Short Alpha-Helices Stabilized by a Main-Chain Hydrogen-Bond Surrogate,” J. Am. Chem. Soc. 128(28):9248-56 (2006), which is hereby incorporated by reference in its entirety).

### Example 4

### Trypsin Digestion Assay

A Tris solution containing 73 μM of tryptophan, 1 ng/μL of trypsin, and 300 μM peptide was incubated at 25° C. At the indicated time intervals, 11 μL of the above solution was quenched with 15 μL of 2% aqueous TFA, and then injected into reversed-phase HPLC to analyze the change in the area of the peptide peak compared to the area of an internal control (tryptophan).

### Example 5

### Binding Assay

Recombinantly purified 6×His-tagged HRas WT 1-166 was incubated with Ni-NTA sepharose beads, generating recombinant Ras on solid support. FITC labeled HBS peptides were incubated in the presence of recombinant HRas-6×His/Ni-NTA Sepharose complex at 4° C. for 1 hour. Beads complexes were pelleted and washed 5× with protein buffer (20 mM Tris-HCl pH 7.6, 50 mM NaCl, 4 mM EDTA, 10 mM MgCl₂), resuspended in reducing buffer (126 mM Tris-HCL pH 6.8, 20% glycerol, 4% SDS, 0.02% bromophenol blue, 2.5% β-mercaptoethanol), boiled for 10 minutes at 95° C., and subjected to SDS-PAGE with an acrylamide percentage of 20% to allow for sufficient resolution of the HBS helices. SDS-PAGE gels were imaged using an ImageQuant™ LAS 4000 biomolecular imager (GE Healthcare, Piscataway, N.J., USA). Quantification of fluorescence by FITC-HBS helices in gel was determined by manufacturer provided ImageQuant™ TL software. Raw values were normalized to total input and plotted on a semilog graph with GraphPad Prism version 6.00 for OSX (GraphPad Software, La Jolla Calif. USA, www.graphpad.com).

### Example 6

### In Vitro Nucleotide Exchange

Exchange assays were performed by incubating 1 μM of recombinant wild type Ras protein with 1 μM mant-GDP in exchange buffer (20 mM Tris-HCl pH 7.6, 50 mM NaCl, 4 mM EDTA) for 5 minutes on ice. The mixture was then supplemented with 10 mM MgCl₂ and incubated for an additional 6 hours at 4° C. Exchange reaction was initiated by either the addition of 100 μM-unlabeled GDP, (negative control) or the addition of 100 μM-unlabeled GDP in the presence of 1 μM SOS-Cat. The levels of fluorescence anisotropy were measured on a Perkin Elmer fluorescence spectrometer (model LS50B) with an excitation wavelength of 370 nm and emission detection at 430 nm. To test the inhibitory properties of the HBS_{SOS} helices, indicated peptides were added to the mant-loaded Ras at a concentration of 1 uM and allowed to stand for 10 minutes at 4° C. before the addition of SosCat. Dissociation rates (K_{off} values) were determined by fitting raw data to a first-order exponential decay function using GraphPad Prism version 6.00 for OSX (GraphPad Software, La Jolla Calif. USA, www.graphpad.com). Data was normalized to t=0, converted to a percentage, and plotted as a curve for ease of visualization.

### Example 7

### Viability Assay with Dose-Response

Various indicated cell lines were plated at low density (˜500-1000 cells per well) in a 96-well format. 12 hours post-plating, media was supplemented with either PBS vehicle, or indicated dosage, and cells were allowed to grow for ˜5.5 days. Cells were subsequently fixed for 1 hour with a 3.7% formaldehyde solution in PBS, and stained with Syto-60 nucleic acid stain (MANUFACTURER) in solution (1:2000 Syto-60, 0.5% Triton X-100, 100 ug/mL RNAse-A, in PBS). Syto-60 stained cells were subjected to quantitative imaging in 96-well plates (Odyssey® Imager, LI-COR Biosciences; excitation: 652; emission: 678) with a 3.0 mm z-axis offset to the scan-head to get the cellular nuclei in the optimal focal plane for quantification. Absolute fluorescence intensity was normalized to the vehicle control for each respective cell line at each dose. Normalized values were visualized on a semilog plot, and data was subjected to non-linear sigmoidal dose-response to determine half-maximal effective concentration (EC₅₀) using GraphPad Prism version 6.00 for OSX, (GraphPad Software, La Jolla Calif. USA, www.graphpad.com).

### Example 8

### Macropinocytic Uptake Assay and EIPA Pretreatment

Cells were plated at ˜50% density on acid-washed coverslips sitting in 12-well format plates. 36 hours later, cells were incubated in respective culture media supplemented with either TMR-conjugated 70 kDa dextran, or FITC-conjugated HBS helices, or both, for 45 minutes. Coverslips were immediately transferred to a 12-well plate format containing 4° Celsius PBS. Coverslips were washed 5 times with full-well volume of 4° Celsius PBS, and fixed with 3.7% formaldehyde for 1 hour. Coverslips were then washed 3 times with full-well volume of 4° Celsius PBS, and then incubated with 1 ug/mL DAPI nuclear stain. Coverslips were then washed 3 times with 4° Celsius PBS, and mounted onto microscope slides with ˜5 uL of fluorescent mounting media (Dako North America, Inc. Carpinteria, Calif.). Quantification was performed utilizing a Zeiss Axiovert 200M inverted epifluorescent microscope (Carl Zeiss Microscopy Thornwood, N.Y.) with a 63× oil objective, and no fewer than 100 cells were imaged in no fewer than 9 fields of view, corresponding to at least 3 individual experiments. Quantification of macropinocytic uptake of fluorescently labeled 70-kDa dextran, or fluorescently labeled HBS helices, was performed in ImageJ 64-bit v1.47 (NIH, USA). Quantification was performed by first subtracting background with a rolling-ball radius of 10-pixels, application of a signal threshold, followed by particle analysis of the entire field-of-view. Total particle area was divided by the number of DAPI positive nuclei, and the raw data values were plotted as macropinocytic uptake. EIPA induced inhibition of macropinocytosis was achieved by preincubation of cell-coated coverslips with 50 uM EIPA for 30 minutes prior to incubation of cells with either or both TMR-70 kDa Dextran, FITC-HBS helices, in cell culture media containing 50 uM EIPA.

### Example 9

### Analysis of DNA Content by FACS

DNA content was determined by flow cytometric analysis using TO-PRO®-3 Iodide (Life Technologies Corp., Grand Island, N.Y.) to stain nucleic acids. Cell lines were plated at ˜40-50% confluency in multi-well format and 12-hours post plating were treated with either HBS helices or vehicle control at the indicated dose. After the indicated incubation period the culture media was collected, and the cells were trypsinized, pooled with the media, pelleted in a clinical centrifuge, resuspended in a small volume of PBS, and added dropwise to 100% ice-cold ethanol while vortexing at low speed, resulting in a final concentration of ˜70% ethanol. Cells were fixed in this solution on ice for 24 hours. Subsequently cells were pelleted at 4° C., washed once with a solution of 1% BSA in PBS, and resuspended in a solution of 1% BSA, 0.5% Triton X-100, 1:5000 TO-PRO®-3 Iodide (Life Technologies Corp., Grand Island, N.Y.). Results and Discussion of Examples 1-9

We previously described a synthetic SOS helix (α-HBS_{SOS}), stabilized by a hydrogen bond surrogate (HBS), that downregulates Ras activation and ERK phosphorylation (Patgiri et al, “An Orthosteric Inhibitor of the Ras-Sos Interaction,” Nat. Chem. Biol. 7(9):585-87 (2011), which is hereby incorporated by reference in its entirety). This HBS SOS helix consisted of α-amino acid residues and targeted Ras with micromolar binding constant and provided dose-dependent modulation of Ras signaling in cell culture. To develop a higher affinity binder for Ras while also improving the proteolytic stability of the peptide helix, we created a non-natural helical construct by careful substitution of natural α-residues with non-natural counterparts. Heterogeneous peptides consisting of α- and β-amino acid residues have been shown to resist proteolytic degradation. In recent studies (Patgiri et al., “Nucleation Effects in Peptide Foldamers,” J. Am. Chem. Soc. 134(28): 11495-502 (2012), which is hereby incorporated by reference in its entirety), we showed that judicious incorporation of a single β-residue per helical turn in HBS peptides affords stable chimeric helices with αααβ (α₃β) repeats, which retain their high affinity for the target receptor. These earlier studies suggested that to preserve the native interactions, side chains that make direct contacts should not be placed on β-residues. We applied the HBS α₃β design strategy to the previously optimized SOS sequence to create α₃β-HBS_{SOS}, FEGiYRLeLLKaEEAN, in which three α-residues from α-HBS_{SOS} have been substituted with β³-amino acids as denoted with lower case letters (FIGS. 4 and 5, and Table 1). We also developed an unconstrained control lacking the HBS macrocycle (α₃β-UNC_{SOS}) and a negative binding control (α₃β-HBS_{MUT}) featuring alanine substitutions for three residues critical for binding (Phe929, Glu942, and Asn944).

TABLE 1
Mass spectroscopic characterization
of α₃β peptides.

| | | | Observed |
|---|---|---|---|
| | | Calculated | MW |
| Number | Sequence | MW | (M + 2)/2 |
| α₃β- | ZFEG*iYRLeLLKaEEAN-NH₂ | 2044.4 | 1022.9 |
| HBS_{SOS} | | | |
| α₃β- | Ac-FEGiYRLeLLKaEEAN-NH₂ | 1978.3 | 989.9 |
| UNC_{SOS} | | | |
| α₃β- | ZAEG*iYRLeLLKaEAAA-NH₂ | 1867.2 | 934.1 |
| HBS_{MUT} | | | |

Lowercase letters depict β³-residues. G* denotes bridged
allylglycine residue. Z = 5-hexenoic acid residue.

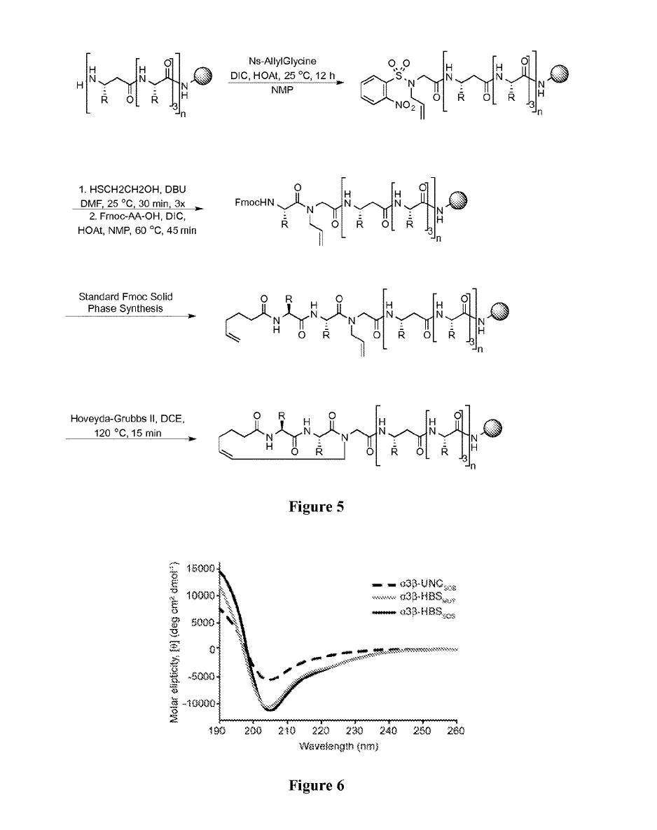

The conformational stability of α₃β-HBS_{SOS} was analyzed using circular dichroism (CD) spectroscopy. The CD traces for α₃β-UNC_{SOS} and α₃β-HBS_{SOS} each feature a minimum near 205 nm and a maximum at 190 nm, which is consistent with previously reported CD spectra of α₃β-HBS helices (FIG. 6). The constrained sequence displays more intense signals, indicative of a more structured peptide.

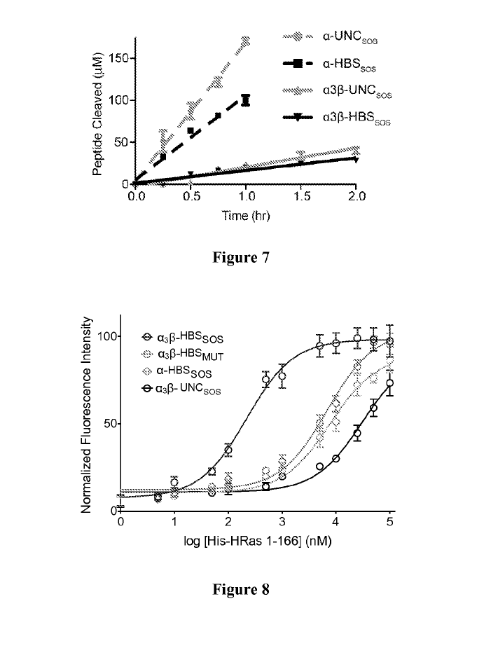

The proteolytic stability of the α3β sequences was assayed in the presence of trypsin (FIG. 7). Trypsin was chosen because the designed sequence contains two cleavage sites for the enzyme following the arginine and lysine residues, allowing monitoring of peptide stability in the context of expected cleavage products. Importantly, the HBS constraint is more than one helical turn away from the lysine residue allowing measurement of the rate of peptide proteolysis without interference from the macrocycle. The initial rate of hydrolysis for the unconstrained all-α peptide sequences ranges from 164.6±9.0 μM/h for the unconstrained sequence (α-UNC_{SOS}: FEGIYRLELLKAEEAN (SEQ ID NO: 5)) to 100.3±6.0 μM/hr for the constrained peptide α-HBS_{SOS}. The α₃β analogs are significantly more stable with cleavage rates of 22.8±1.9 μM/hr and 15.0±1.3 μM/hr for α₃β-UNC_{SOS} and α₃β-HBS_{SOS}, respectively.

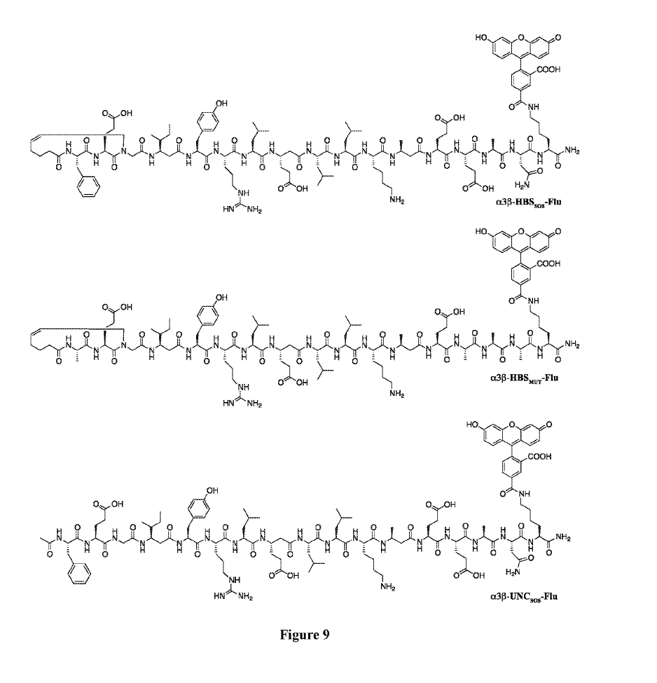

The binding affinity of the designed compounds for Ras was determined using in vitro pulldown assays with recombinantly purified 6×His-tagged HRas⁽¹⁻¹⁶⁶⁾ and fluorescein-tagged SOS peptides (FIGS. 8 and 9, and Table 2). We found that α-HBS_{SOS} binds to HRas⁽¹⁻¹⁶⁶⁾ with affinity (K_{D}=7.75±3.0 uM) comparable to previously reported values obtained using a fluorescence polarization assay. α₃β-HBS_{SOS} binds to HRas (K_{D}=220±56 nM) with 50-fold greater affinity than the all-α analog α-HBS_{SOS}. The unconstrained analog α₃β-UNC_{SOS} (K_{D}=30.6±12 uM) displays a ˜136-fold weaker affinity than its constrained counterpart α₃β-HBS_{SOS}, reflecting the extent of preorganization endowed by the HBS constraint. Although, the exact binding mode of the peptides and the conformational state of Ras is not known, the enhanced affinity of the α₃β-HBS_{SOS} compound likely reflects a combination of both the higher conformational stability of the HBS α₃β construct and the exact Ras conformation it can access. In previous studies, we showed that the HBS SOS mimics bind to both nucleotide-free and nucleotide-bound Ras, but to the free Ras with higher affinity. Because, the SOS analogs can bind nucleotide-free and bound Ras, we hypothesize that the compounds are accessing an intermediate switch conformation.

TABLE 2
Mass spectroscopic characterization of fluorescein-labeled α₃β peptides.

| | | Calculated | Observed |
|---|---|---|---|
| Number | Sequence | MW | MW (M + 2)/2 |
| α3β-HBS_{SOS}-Flu | ZFEG*iYRLeLLKaEEANK(Flu)-NH₂ | 2530.8 | 1266.0 |
| α3β-UNC_{SOS}-Flu | Ac-FEG*iYRLeLLKaEEANK(Flu)-NH₂ | 2464.7 | 1233.0 |
| α3β-HBS_{MUT}-Flu | ZAEG*iYRLeLLKaEAAAK(Flu)-NH₂ | 2353.7 | 1177.9 |

Lowercase letters depict β³-residues. G* denotes bridged allylglycine residue.
Z = 5-hexenoic acid residue.

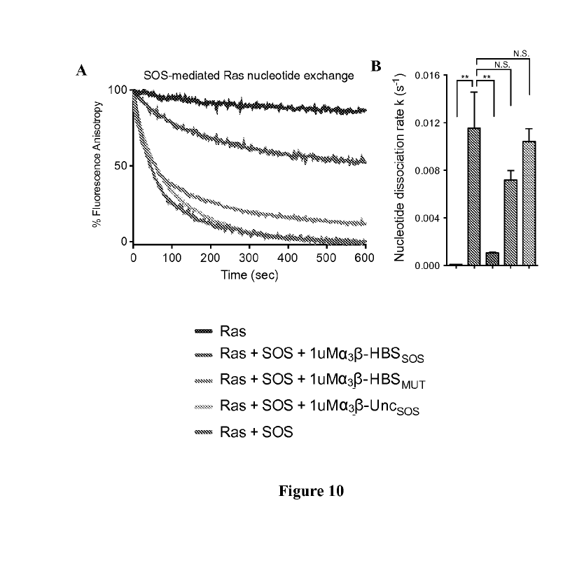

We next analyzed the potential of the mimetics to inhibit Sos-mediated Ras nucleotide exchange in a cell-free system. α₃β-HBS_{SOS} has a potent inhibitory effect compared to both α₃β-UNC_{SOS} and α₃β-HBS_{MUT} (FIG. 10). We evaluated inhibition of nucleotide exchange under stringent conditions consisting of 1 μM each of Ras, SOS, and the inhibitor. The inhibitory effect of α₃β-HBS_{SOS} was comparable to that of the previously reported α-HBS_{SOS}, but at one-twentieth the concentration, further supporting the enhanced stability and binding affinity of α₃β-HBS_{SOS}.

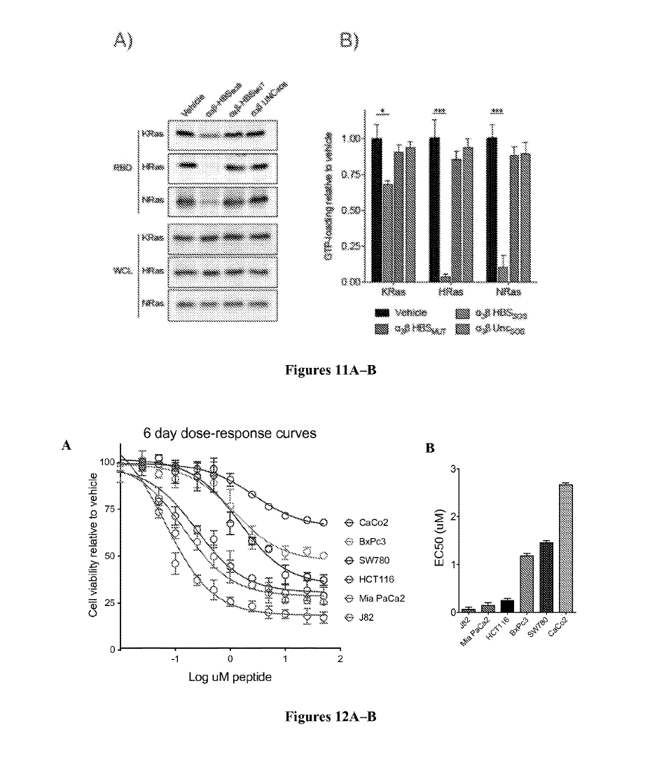

To determine the effect of α₃β-HBS_{SOS} on Sos-mediated Ras activation in cells we treated MIA PaCa-2 cells, which harbor oncogenic KRas, with α₃β-HBS_{SOS}, control peptides, or vehicle control for 48 hours. Utilizing the RBD-pulldown assay we determined the levels of GTP-loaded endogenous HRas, KRas, and NRas proteins. As expected, MIA PaCa-2 cells display GTP-loading of all isoforms at steady state when treated with the vehicle control (FIGS. 11A-B). Treatment with α₃β-HBS_{SOS}, but not α₃β-UNC_{SOS} or α₃β-HBS_{MUT}, completely abrogated the GTP-loading of wild type HRas and NRas isoforms, indicating potent in-cell inhibitory effects on Sos-mediated Ras nucleotide exchange. Significantly, α₃β-HBS_{SOS} diminished the GTP-loading of the oncogenic isoforms supporting the recent findings that oncogenic Ras requires Sos-mediated nucleotide exchange to maintain constitutive activation. Taken together these observations suggest that α₃β-HBS_{SOS} is a proteolytically and conformationally stable helix mimic that can inhibit Sos-mediated Ras activation in cancer cells.

To investigate the functional consequence of inhibiting Ras activation in the context of oncogenic mutations, we examined the effect of α₃β-HBS_{SOS} on the growth and viability of cancer cells of different origins. We selected a panel of six human cancer cell lines derived from the pancreas, colon, and bladder, which harbor a variety of oncogenic proteins including mutant KRas (Table 3). Treatment of cells with a single administration of 5 μM of α₃β-HBS_{SOS} for six days inhibited the growth of all cell lines to varying degrees relative to vehicle control (FIGS. 12A-B). In contrast treatment with α₃β-HBS_{MUT}, α₃β-UNC_{SOS}, or α-HBS_{SOS} had no effect on cell growth at these concentrations, indicating, respectively, the importance of the primary sequence, HBS constraint, enhanced binding affinity, and proteolytic stability. Notably, the viability of cell lines harboring oncogenic KRas were significantly more affected than cell lines wild type for Ras. The sole exception was the J82 cell line, which harbors constitutively active FGFR3, suggesting that inhibition of Ras activation is broadly applicable to cancer cells with Ras-dependent genetic signatures.

α₃β-HBS_{SOS} reduces cell viability in a dose dependent manner, with the effect spanning over four orders of magnitude. We divided the cell lines into two groups with respect to EC₅₀ values (Table 3). The ‘oncogenic RTK-Ras’ group, containing MIA PaCa-2, HCT-116, and J82 cells, were remarkably sensitive to α₃β-HBS_{SOS} treatment (EC₅₀=144±57 nM, 243±44 nM, and 65.2±43 nM respectively). This is in contrast to the wild-type Ras group, containing CaCo2, SW780, and BxPc3, which exhibited limited effects on viability in response to α₃β-HBS_{SOS} treatment. This data implies that RTK-Ras mutational status indicates sensitivity to α₃β-HBS_{SOS}. Interestingly, at increasing doses of α₃β-HBS_{SOS} the viability of SW780, BxPc3 and CaCo2 cells is moderately affected. We hypothesized that differential cellular uptake may account for the different potency of the peptides between the oncogenic and wild type RTK-Ras cell lines.

TABLE 3
Summary of Ras mutational state for tested cell lines and the EC50 values
for cell viability in response to treatment with α₃β-HBS_{SOS}.

| | EC50 (μM) | Ras status | Mutations |
|---|---|---|---|
| CaCo2 | 2.674 | WT | BRAF |
| BxPc3 | 1.182 | WT | VEGF-c |
| SW780 | 1.453 | WT | NRG1, FGFR3, IGF1R |
| HCT116 | 0.2434 | Kras G13C | Pi3K |
| Mia PaCa2 | 0.1441 | Kras G12D | NF1 |
| J82 | 0.0652 | WT | FGFR3 CA, Pi3K, PTEN |

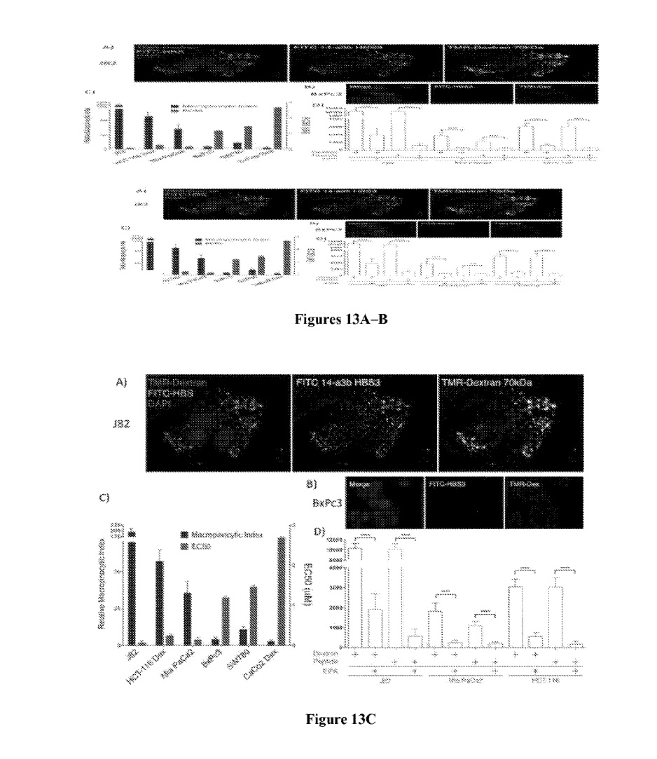

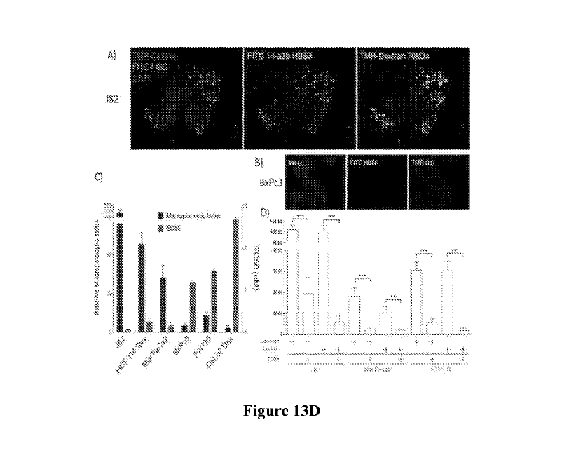

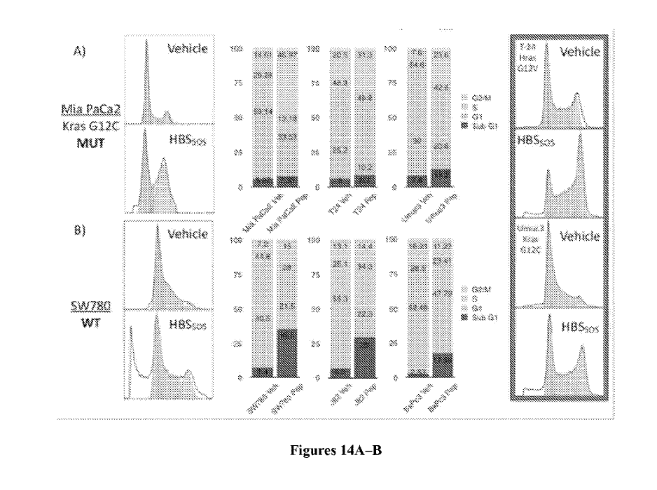

Constitutively active Ras and RTK signaling stimulate membrane ruffling and macropinocytosis. This indiscriminate internalization of extracellular fluid and protein is required by Ras-driven cancer cells as an amino acid supply route to supplement their growth. Therefore, we hypothesized that would likewise be internalized by macropinocytosis, and moreover reasoned that its proteolytic stability would afford it the capacity to persist within a degradative compartment. In order to determine the extent to which α₃β-HBS_{SOS} undergoes macropinocytic uptake, we incubated cell lines simultaneously with fluorescently-labeled TMR 70-kDa Dextran, a specific marker of macropinosomes, and 5FAM α₃β-HBS_{SOS}. Laser scanning confocal microscopy (LSCM) confirmed a high degree of colocalization, indicating that the vast majority of α₃β-HBS_{SOS} internalization occurs through macropinocytosis (FIGS. 13A-B). Furthermore the uptake of both 5FAM-labeled α₃β-HBS_{SOS} and TMR-labeled 70-kDa Dextran was abolished by pretreatment of cells with EIPA, an inhibitor of macropinocytosis (FIG. 13C). We calculated the relative levels of macropinocytic uptake by our panel of cell lines with quantitative imaging of TMR 70-kDa Dextran (FIG. 13D). Increasing macropinocytic uptake correlated inversely with the half maximum effective dose (EC50) on cell viability, indicating that Ras-stimulated macropinocytosis facilitated internalization of α₃β-HBS_{SOS}. Taken together, this data identifies macropinocytosis as a measure of sensitivity to α₃β-HBS stabilized helices.

We next sought to gain mechanistic insight into the attenuated growth and viability of cancer cells that is observed upon inhibition of Sos-mediated Ras activation with α₃β-HBS_{SOS}. Because of the link between Ras signaling and cell cycle progression, we reasoned that DNA content analysis would elucidate Ras-mediated effects on cell growth. Flow cytometric analysis of the cell cycle revealed an accumulation of mutant Ras cells (MIA PaCa-2, HCT-116, Umuc3, and T24) with 4N DNA content upon treatment with α₃β-HBS_{SOS} for 48 hours (FIG. 14A). In contrast, the cell lines with wild type for Ras (SW780, BxPc3, J82) do not accumulate in the G2/M phase of the cell cycle (FIG. 14B). Furthermore, only mutant Ras cells displayed increased levels of both DNA strand breaks and apoptosis, as determined by quantification of phosphorylated H2AX and cleaved caspace-3 immunofluorescence, respectively (FIGS. 14A-B). This cellular response to pharmacological inhibition of Ras activation by α₃β-HBS_{SOS} phenocopies the effects of the post-transcriptional silencing of wild type Ras and Sos in oncogenic Ras-driven cancer. These corroborant genetic and pharmacological cell viability defects support the therapeutic strategy of targeting wild type Ras activation.

Sos-mediated hyperactivation of wild type Ras is an ineluctable outcome of constitutively active Ras and RTK activity, and a critical aspect of the Ras-transformed phenotype. The present study identifies Ras-stimulated macropinocytic uptake of synthetic α-helix mimics of Sos as a strategy for the cancer-specific therapeutic targeting of Ras activation.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A peptide a peptide having a stable, internally-constrained HBS α-helix, where the peptide mimics at least a portion of the α-H helix of the Sos protein and contains a mixture of alpha and beta amino acid residues in the pattern α3/β1.

2. The peptide according to claim 1, wherein the peptide comprises a sequence of formula (X/Z)-AA¹-AA²-AA³-AA⁴-AA⁵-AA⁶-AA⁷-AA⁸-AA⁹-AA¹⁰-AA¹¹-AA¹²-AA¹³-AA¹⁴-AA¹⁵-AA¹⁶, wherein X is 4-pentenoic acid; Z is 5-hexenoic acid; AA¹-AA¹⁶ are each independently an alpha or beta amino acid residue; AA¹ is Phe; AA² is any amino acid residue; AA³ is Gly or Ala; AA⁴ is any amino acid residue; AA⁵ is any charged (preferably positively) and/or aromatic amino acid residue; AA⁶ is any amino acid residue; AA⁷ is an amino acid residue that is hydrophobic and aliphatic or able to form a hydrogen bond; AA⁸ is any amino acid residue; AA⁹ is any amino acid residue; AA¹⁰ is Leu or any charged amino acid residue; AA¹¹ is any amino acid residue; AA¹² is any amino acid residue; AA¹³ is any charged amino acid residue; AA¹⁴ is any amino acid residue; AA¹⁵ is any amino acid residue); AA¹⁶ is Asn; and * denotes the placement of the internal constraint (i.e., between (X/Z) and AA³).

3. The peptide according to claim 2, wherein AA¹ is Phe; AA² is Glu or Asp; AA³ is Gly or Ala; AA⁴ is any amino acid residue; AA⁵ is Tyr, Phe, Trp, Arg, or Lys; AA⁶ is Arg or Lys; AA⁷ is Leu, Ile, Val, Thr, or Ser; AA⁸ is Glu, Asp, Gin, Asn, Arg, or Lys; AA⁹ is any amino acid residue; AA¹⁰ is Leu, Arg, Lys, His, Glu, or Asp; AA¹¹ is Lys or Arg; AA¹² is any amino acid residue; AA¹³ is Glu, Asp, Lys, or Arg; AA¹⁴ is Glu; AA¹⁵ is Ala or Gly; and AA¹⁶ is Asn.

4. The peptide according to claim 3, wherein the sequence is selected from the group consisting of (X/Z)FEG*iYRLeLLKaEEAN, (X/Z)FEg*IYRlELLkAEEaN, XFeG*IYrLELlKAEeAN, XfEG*IyRLElLKAeEAN, XFEG*iYRLeLLKaEEAN, ZFEG*iYRLeLLKaEEAn, ZFEG*iYRTeLLKaEEAN, ZFEG*iYRLqLLKaEEAN, ZFEg*IYRlELLkAEEaN, XFEg*IYRlELLkAEEaN, ZFEg*IYRtELLkAEEaN, ZFEg*IYRlQLLkAEEaN, XFeG*IYrTELlKAEeAN, XFeG*IYrLQLlKAEeAN, XfEG*IyRTElLKAeEAN, and XfEG*IyRLQlLKAeEAN.

5. The peptide according to claim 1, wherein the peptide is a peptide of Formula I: 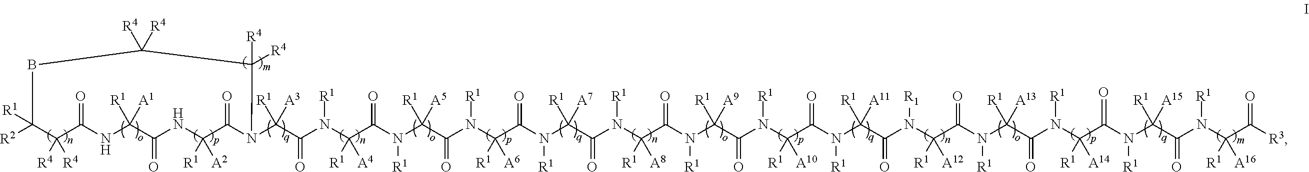
wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
m is one or two;
each n is the same and is one or two;
each o is the same and is one or two;
each p is the same and is one or two; and
each q is the same and is one or two;
wherein at least one of the following conditions is met
(i) n, o, and p are one and q is two;
(ii) n, o, and q are one and p is two;
(iii) n, p, and q are one and o is two;
(iv) o, p, and q are one and n is two.

6. The peptide according to claim 5, wherein the peptide comprises a sequence of formula (X/Z)-AA¹-AA²-AA³*-AA⁴-AA⁵-AA⁶-AA⁷-AA⁸-AA⁹-AA¹⁰-AA¹¹-AA¹²-AA¹³-AA¹⁴-AA¹⁵-AA¹⁶, wherein X is 4-pentenoic acid; Z is 5-hexenoic acid; AA¹-AA¹⁶ are each independently an alpha or beta amino acid residue; AA¹ is Phe; AA² is any amino acid residue; AA³ is Gly or Ala; AA⁴ is any amino acid residue; AA⁵ is any charged (preferably positively) and/or aromatic amino acid residue; AA⁶ is any amino acid residue; AA⁷ is an amino acid residue that is hydrophobic and aliphatic or able to form a hydrogen bond; AA⁸ is any amino acid residue; AA⁹ is any amino acid residue; AA¹⁰ is Leu or any charged amino acid residue; AA¹¹ is any amino acid residue; AA¹² is any amino acid residue; AA¹³ is any charged amino acid residue; AA¹⁴ is any amino acid residue; AA¹⁵ is any amino acid residue); AA¹⁶ is Asn; and * denotes the placement of the internal constraint (i.e., between (X/Z) and AA³).

7. The peptide according to claim 6 wherein AA¹ is Phe; AA² is Glu or Asp; AA³ is Gly or Ala; AA⁴ is any amino acid residue; AA⁵ is Tyr, Phe, Trp, Arg, or Lys; AA⁶ is Arg or Lys; AA⁷ is Leu, Ile, Val, Thr, or Ser; AA⁸ is Glu, Asp, Gin, Asn, Arg, or Lys; AA⁹ is any amino acid residue; AA¹⁰ is Leu, Arg, Lys, His, Glu, or Asp; AA¹¹ is Lys or Arg; AA¹² is any amino acid residue; AA¹³ is Glu, Asp, Lys, or Arg; AA¹⁴ is Glu; AA¹⁵ is Ala or Gly; and AA¹⁶ is Asn.

8. The peptide according to claim 7, wherein the sequence is selected from the group consisting of (X/Z)FEG*iYRLeLLKaEEAN, (X/Z)FEg*IYRlELLkAEEaN, XFeG*IYrLELlKAEeAN, XfEG*IyRLElLKAeEAN, XFEG*iYRLeLLKaEEAN, ZFEG*iYRLeLLKaEEAn, ZFEG*iYRTeLLKaEEAN, ZFEG*iYRLqLLKaEEAN, ZFEg*IYRlELLkAEEaN, XFEg*IYRlELLkAEEaN, ZFEg*IYRtELLkAEEaN, ZFEg*IYRlQLLkAEEaN, XFeG*IYrTELlKAEeAN, XFeG*IYrLQLlKAEeAN, XfEG*IyRTElLKAeEAN, and XfEG*IyRLQlLKAeEAN.

9. The peptide according to claim 1, wherein the peptide is selected from the group consisting of (X/Z)FEG*iYRLeLLKaEEAN-NH₂, (X/Z)FEg*IYRlELLkAEEaN-NH₂, XFeG*IYrLELlKAEeAN-NH₂, XfEG*IyRLElLKAeEAN-NH₂, XFEG*iYRLeLLKaEEAN-NH₂, ZFEG*iYRLeLLKaEEAn-NH₂, ZFEG*iYRTeLLKaEEAN-NH₂, ZFEG*iYRLqLLKaEEAN-NH₂, ZFEg*IYRlELLkAEEaN-NH₂, XFEg*IYRlELLkAEEaN-NH₂, ZFEg*IYRtELLkAEEaN-NH₂, ZFEg*IYRlQLLkAEEaN-NH₂, XFeG*IYrTELlKAEeAN-NH₂, XFeG*IYrLQLlKAEeAN-NH₂, XfEG*IyRTElLKAeEAN-NH₂, and XfEG*IyRLQlLKAeEAN-NH₂.

10. A pharmaceutical composition comprising a peptide according to claim 1 and a pharmaceutically acceptable vehicle.

11. A pharmaceutical composition comprising a peptide according to claim 2 and a pharmaceutically acceptable vehicle.

12. A pharmaceutical composition comprising a peptide according to claim 5 and a pharmaceutically acceptable vehicle.

13. A method of inhibiting Ras signaling in a cell, the method comprising:
contacting the cell with a peptide according to claim 1 under conditions effective to inhibit Ras signaling in the cell.

14. A method of inhibiting Ras signaling in a cell, the method comprising:
contacting the cell with a peptide according to claim 2 under conditions effective to inhibit Ras signaling in the cell.

15. A method of inhibiting Ras signaling in a cell, the method comprising:
contacting the cell with a peptide according to claim 5 under conditions effective to inhibit Ras signaling in the cell.

16. A method of promoting cell death, the method comprising:
contacting the cell with a peptide according to claim 1 under conditions effective for the peptide to promote cell death.

17. A method of promoting cell death, the method comprising:
contacting the cell with a peptide according to claim 2 under conditions effective for the peptide to promote cell death.

18. A method of promoting cell death, the method comprising:
contacting the cell with a peptide according to claim 5 under conditions effective for the peptide to promote cell death.

19. A method of treating, preventing, and/or diagnosing a cellular proliferative disorder, differentiative disorder, and/or neoplastic condition in a subject in need thereof, the method comprising:
administering to the subject a composition comprising a peptide according to claim 1.

20. The method according to claim 19, wherein the cellular proliferative disorder, differentiative disorder, and/or neoplastic condition is selected from the group consisting of fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi sarcoma; hematopoietic neoplastic disorders; cellular proliferative and/or differentiative disorders of the breast; lar proliferative and/or differentiative disorders of the lung; cellular proliferative and/or differentiative disorders of the colon; cellular proliferative and/or differentiative disorders of the liver; cellular proliferative and/or differentiative disorders of the ovary; a cancer mediated by a mutated Ras protein; and immunoproliferative disorders.

21. The method according to claim 20, wherein the cellular proliferative disorder, differentiative disorder, and/or neoplastic condition is pancreatic cancer, colon cancer, or bladder cancer.

22. The method according to claim 19, wherein the peptide is administered under conditions effective to treat or prevent a cellular proliferative disorder, differentiative disorder, and/or neoplastic condition in the subject.

23. A method of treating, preventing, and/or diagnosing a cellular proliferative disorder, differentiative disorder, and/or neoplastic condition in a subject in need thereof, the method comprising:
administering to the subject a composition comprising a peptide according to claim 2.

24. A method of treating, preventing, and/or diagnosing a cellular proliferative disorder, differentiative disorder, and/or neoplastic condition in a subject in need thereof, the method comprising:
administering to the subject a composition comprising a peptide according to claim 5.


SEQUENCE LISTING (5 sequences)

SEQ ID NO: 1 - protein, 29 aa, Artificial
  Signal Peptide
MMSFVSLLLVGILFYATEAEQLTKCEVFQ

SEQ ID NO: 2 - protein, 4 aa, Artificial
  Endoplasmic Reticulum Retention Signal
KEDL

SEQ ID NO: 3 - protein, 8 aa, Artificial
  Nuclear Transport Peptide
PPKKKRKV

SEQ ID NO: 4 - protein, 25 aa, Artificial
  Transport Peptide for Targeting to the Mitochondria
MLSLRQSIRFFKPATRTLCSSRYLL

SEQ ID NO: 5 - protein, 16 aa, Artificial
  Unconstrained Peptide
FEGIYRLELLKAEEAN